(19) United States
(12) Patent Application Publication
(10) Pub. No.: US 2015/0093840 A1
(43) Pub. Date: Apr. 2, 2015

(54) ENZYME-FREE COLORIMETRIC IMMUNOASSAY

(71) Applicant: NATIONAL TSING HUA UNIVERSITY, Hsinchu (TW)

(72) Inventors: Jing-Huei HUANG, Hsinchu (TW); Ying-Chan HUNG, Hsinchu (TW); Tri-Rung YEW, Hsinchu (TW); Pin CHANG, Hsinchu (TW)

(73) Assignee: NATIONAL TSING HUA UNIVERSITY, Hsinchu (TW)

(21) Appl. No.: 14/098,116

(22) Filed: Dec. 5, 2013

(30) Foreign Application Priority Data
Sep. 27, 2013 (TW) 102134968

(51) Int. Cl. G01N 33/58

(52) U.S. Cl. CPC G01N 33/583
USPC 436/501

(57) ABSTRACT

A colorimetric immunoassay of the present invention uses nanostructured material with high absorption and high scattering ability as a label material for biosensors. Subject matters to be measured may be characterized or quantified by determining the changes in optical properties of the nanostructured material. The biosensor of the present invention may be operated in broad light wavelength range and detected by direct observation with naked eye. The biosensor of the present invention may be also provided with advantages such as higher sensitivity and lower cost.

### BACKGROUND OF THE INVENTION

1. Field of the Invention

The present invention relates to a colorimetric immunoassay, particularly relates to an enzyme-free colorimetric immunoassay.

2. Description of the Prior Art

Immunoassay biosensors based on the specificity of antigen-antibody recognition reaction have become more and more important in clinical diagnostics and treatment. Nowadays, a large number of studies have been focused on using the unique properties of nanomaterials to develop various novel nanostructure-based immunoassay biosensors such as electromechanical, electrical and mass-sensitive biosensor systems. These biosensors exhibit high sensitivity, but the incorporated expensive sensing systems still limit their applications in daily life.

One way to solve this issue is using ultraviolet-visible spectroscopy (UV-Vis) as the sensing system for optical biosensors since it takes no complicated instrument to output detecting signals so as to lower the cost of biosensors and be applicable in daily life with great potential.

### ELISA (Enzyme-Linked Immunosorbent Assay)

ELISA has been a traditional bio-sensing method and now been developed into commercially available detection kit used for medical testing. ELISA, based on the specificity characteristics between antigen-antibody and used for in vitro testing, can be used in conjunction with the enzymatic colorimetric reaction to show the existence of a particular antigen or antibody and achieve quantitative analysis by color depth.

In consideration of various samples and bonding mechanisms, ELISA methods are mainly categorized into sandwich method, indirect method, as well as competitive method. There have been many commercially available kits for various antigens and antibodies; however, there exist some drawbacks such as higher cost, more complicated procedures and some professional training required for the testing personnel.

### Gold Nanoparticles Used in Immune Biosensor

Due to special optical properties in connection to very good biocompatibility, gold nanoparticles (AuNPs) are now most common materials applied in the colorimetric biosensors. When the size of AuNPs is reduced to the nanometer scale, namely less than the wavelength of visible light, it results in generating very strong optical absorption properties because of the size and shape effect. This is because outer electrons of gold particles are susceptible to electromagnetic radiation to generate periodically oscillation. Dramatic color change characteristics of AuNPs, i.e. AuNPs are red when dispersed and blue to purple when aggregated, can be used for analysis in a quick manner. Examples such as binding of AuNPs to the protein or gene sequence used in immune assay are very popular research topics, currently. Although AuNPs are provided with obvious color change and can be applied in colorimetric biosensors, AuNPs have disadvantages such as higher cost and results in increased cost for biosensors.

### Application of Carbon Nanotubes in Biosensors

Carbon nanotubes, provided with features such as good mechanical, electrical and electrochemical properties, as well as high surface area to volume ratio, and good biocompatibility, have been widely used in electronics, optoelectronics and biological fields. The current biological applications for carbon nanotubes mainly focus in drug release and protein and DNA-based biosensors. In recent years, carbon nanotubes have also been applied in colorimetric biosensors in some studies. 2007, Lee et al (Nanotechnology, 2007, 18, 455102-455120) used carbon nanotubes carrying HRP to nucleic acid by using the colorimetric effect of carbon nanotubes caused by aggregation.

In addition, Song et al (Chem. Eur. J., 2010, 16, 3617-3621.) reported in 2010 that carbon nanotubes are provided with peroxidase-like activity and may catalyze the reaction of peroxidase substrate in the presence of hydrogen peroxide to produce a color change to detect SNP (single nucleotide polymorphisms).

To sum up, it is now the current goal to develop a low-cost, convenient, and fast biosensor that can be directly detected and identified by the naked eye so as to achieve rapid screening purposes.

### SUMMARY OF THE INVENTION

One purpose of the present invention is directed to develop a low-cost, convenient and fast-detecting biosensor, which can be identified with color change so as to achieve test purposes by naked eye and further quantitative analysis by UV/VIS absorption spectroscopy.

According to one embodiment of the present invention, an enzyme-free colorimetric immunoassay used for detecting an antigen, comprising providing a colorimetric antibody, wherein the colorimetric antibody is coupled with a nanomaterial and not connected with an enzyme and the nanomaterial is black; and measuring the colorimetric effect of the colorimetric antibody so as to determine the presence or concentration of the antigen.

Other advantages of the present invention will become apparent from the following descriptions taken in conjunction with the accompanying drawings wherein certain embodiments of the present invention are set forth by way of illustration and examples.

### BRIEF DESCRIPTION OF THE DRAWINGS

The foregoing aspects and many of the accompanying advantages of this invention will become more readily appreciated as the same becomes better understood by reference to the following detailed descriptions, when taken in conjunction with the accompanying drawings, wherein:

FIGS. 1a to 1c are schematic diagram illustrating a biological sensor in an embodiment of the present invention;

FIGS. 2a to 2d are schematic diagrams and fluorescence photos illustrating successful fixation of mAHSA and blocking of BSA on APTES-modified glass in an embodiment of the present invention;

FIGS. 3a to 3d are schematic diagrams and fluorescence photos illustrating successful conjugation of HSA and mAHSA on the sense substrate in an embodiment of the present invention;

FIG. 4a is an SEM image illustrating the COOH-modified carbon nanotubes in an embodiment of the present invention;

FIG. 4b is an image illustrating the dispersion of COOH-modified CNTs in a buffer solution after standing for 24 hours in an embodiment of the present invention;

FIGS. 4c to 4d are schematic diagrams and fluorescence photos illustrating the combination of anti-IgG-FITC antibody and CNT-label having pAHSA in an embodiment of the present invention;

FIG. 5a illustrates combination of different concentrations of HSA and the sensing substrate with CNT-label in an embodiment of the present invention;

FIG. 5b illustrates transmittance of the sense substrate applied with concentrations of HSA at a wavelength of 400 nm in an embodiment of the present invention; and

FIG. 6 illustrates the combination of different concentrations of HSA and the sense substrate with CNT-label in an embodiment of the present invention.

### DESCRIPTION OF THE PREFERRED EMBODIMENT

The colorimetric immunoassay of the present invention method is used for detecting an antigen. The present invention provides the use of nanomaterials with high absorption coefficient to form nanostructures with highly scattering ability and can be developed into a novel biosensor. The colorimetric antibody of the present invention is an antibody conjugated with high absorption nanomaterials, which replace the rule of the enzyme played in the enzyme-linked immunosorbent assay and achieve colorimetric effect without needs of enzyme combination. The colorimetric immunoassay of the present invention may be achieved by measuring the colorimetric effect of the colorimetric antibody to determine the presence or concentration of the antigen, thus achieving testing purposes.

### Nanomaterials

The present invention adopts nanomaterials with high absorption coefficient, preferably being black. Examples of applicable nanomaterials may include but not be limited to CNTs (carbon nanotubes), graphene, cobalt oxide (Co₃O₄) or tungsten disulfide (WS₂). Here, CNTs may include SWCNTs (single-walled carbon nanotubes) or MWCNTs (multi-walled carbon nanotubes). Graphene may also include graphene oxides.

In principle, the nanomaterials of the present invention have no specific restriction in other physical properties. However, in the design of nanostructures with high absorption coefficient, certain physical parameters of the nanomaterials may be optimized, for example, selecting nanomaterials by size, shape and composition in order to get desired effect.

In addition, the nanomaterials of the present invention may be modified to achieve the desired properties. Modification methods for nanomaterials may be various, and also be well known to those skilled. Generally, once the surface of the material has been modified with activated amino groups, it is ready for conjugation with the antibody. In one embodiment, the colorimetric antibody of the present invention may be prepared by using nanomaterials of the present invention surface-modified with COOH and underwent chemical modification of EDC (1-ethyl-3-(3-dimethylaminopropyl)carbodiimide) and NHS(N-hydroxysuccinimide) other examples such as Co₃O₄ nanomaterial may be modified to include OH group through acid modification, and then modified with APTES (3-aminopropyl triethoxy silane (3-aminopropyltriethoxysilane) to include activated amino group, which is ready to be bound to the antibody.

Thanks to the non-resonant property, the carbon nanotube structure provides a broad range of light absorption band and are also reported in the literature to have a high absorption coefficient as its optical properties of (a=2.4×10⁵ cm⁻¹). CNT also can increase the light absorption because of high scattering characteristics provided in nanomaterials. In addition, the surface modification for carbon nanotube is very easy for performing conjugation to biological molecules using simple chemical reactions. As commercial carbon nanotubes become more popular now, the cost of carbon nanotubes has dropped significantly. Therefore, the present invention uses carbon nanotubes to verify the feasibility of this novel biosensor and looks forward to daily use in the future.

### Immunoassay

The immunoassay methods of the present invention include, but are not limited to, ELISA, immune complex test, protein microarray analysis, immunoprecipitation, immunochromatography, and so on. One or more of those immunoassay methods are non-invasive, and require minimal or no other apparatuses. The basic implementation of immunological reagents may be referred in technical books and manuals regarding immunoassays.

As for ELISA, in consideration of various samples and bonding mechanisms, ELISA methods mainly are categorized into sandwich method, indirect method, as well as competitive method Sandwich method is commonly used in the detection of macromolecular antigens; indirect method is commonly used in the detection of antibodies, and competitive method is generally used to detect small molecule antigen and is a less used detection mechanism.

As mentioned above, the nanostructures of high absorption coefficient may replace the rule played by the enzyme in ELISA to achieve colorimetric effect. In one embodiment, the colorimetric antibody the present invention binds to the antigen and is applied in sandwich method or indirect method. Alternatively, in another embodiment, the colorimetric antibody of the present invention is applied to sandwich method or the competitive method and acts as a secondary antibody, which binds to the primary antibody, and the primary antibody binds to the antigen.

The present invention may be operated on a solid support, as long as the colorimetric effect of the nanostructures can be observed. For example, the present invention may be operated on a white solid support, and then observed for the change in absorbance spectrum. The solid support can be added with some structures above, for example, 3D structure defined and generated by mask, so as to increase the sensitivity of detection.

In a preferred embodiment, the present invention is operated on a transparent substrate, for example but not limited to ELISA plate made of PS (polystyrene) or a glass substrate. When operated on a transparent substrate, the present invention can measure the colorimetric effect by the measurement of the transmittance changes of the transparent substrate, and can be achieved by an optical microscope or a UV/Vis spectrometer. The measurement wavelength can be performed in visible, near-infrared light with wavelength ranging from but not limited to 400-800 nm and so on. Otherwise, in a preferred embodiment, the colorimetric effect of the colorimetric antibody could be identified by the naked eye so as to achieve the effect of rapid detection.

A standard group may also be created as performed in a common colorimetric test, and the detection result may be compared to color standards of the standard group to determine the corresponding concentration and reach quantitative purposes.

The present invention is further illustrated by the following working examples, which should not be construed as further limiting.

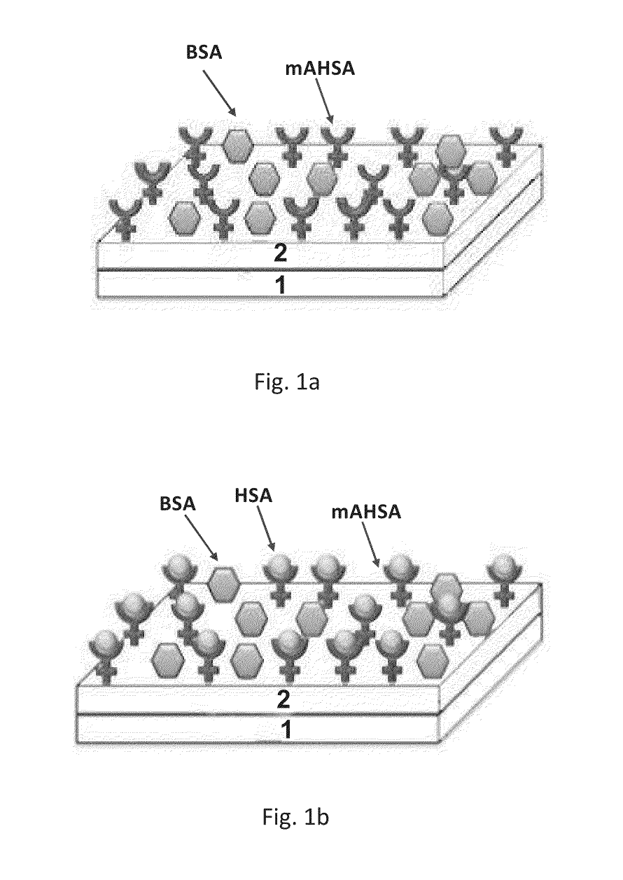

Referring to FIGS. 1a to 1c, the design concept of the biosensor according to an embodiment of the present invention is described as follows. First of all, mAHSA (anti-human serum albumin, monoclonal antibody) was bonded to the sensing-substrate with BSA (bovine serum albumin) as a blocking agent to specifically identify the analyte, HSA (human serum albumin). The analyte HSA is immobilized on the sensing-substrate and then CNT-label immobilized with pAHSA (anti-human serum albumin, polyclonal antibody) was added, where pAHSA of CNT-label was configured for identifying the analyte HSA that have been bound to mAHSA and CNT are configured for providing optical absorption signals. The colorimetric effects caused by different concentration of the analyte are identified with naked eyes and further supplemented with measuring the transmittance of the biosensor by UV/VIS absorption spectroscopy for quantitative analysis so as to confirm the test results achieved by the method of the present invention with naked-eye.

### Fabrication of the Sensing-Substrate

First, the glass substrate was functionalized with hydroxyl groups by piranha solution (1:3 H₂O₂-concentrated H₂SO₄). The amino-group layer of APTES was self-assembled on the glass for 2 h. The glass was then rinsed with DI water for several times, dried by N₂ flow, and baked at 120° C. for 30 min to form a stable APTES film. Second, the APTES-modified glass was then incubated in 0.1 M phosphate buffered saline (PBS) containing 8 μg ml⁻¹ of mAHSA and shaken at 35° C. for 1 h. Third, the mAHSA/APTES-modified glass was incubated in 1 wt % BSA and shaken at 35° C. for 1 h to block untreated and non-specific sites. Moreover, the specimen was washed with PBS for several times after each step of process.

### Fabrication of the CNT-Label

The pAHSA was covalently bound on CNTs with 1-ethyl-3-(3-dimethylaminopropyl)carbodiimide (EDC) and N-hydroxysuccinimide (NHS) reaction, and the detailed fabrication process is described as follows. First, the 0.12 g L⁻¹ carboxylic group modified with CNTs (COOH-modified CNTs) (Golden Innovation Business, CDH-AMC SW2012) in DI water were prepared under ultra-sonication for at least 30 min. Second, the 0.5 ml COOH-modified CNTs were mixed with 0.5 ml 0.1 M buffer solution, i.e., the PBS with KH₂PO₄ (0.2 g L⁻¹) and Na₂HPO₄ (1.16 g L⁻¹), containing 250 mM 1-ethyl-3-(3-dimethylaminopropyl)carbodiimide (EDC, Alfa Aesar), and 100 mM N-hydroxysuccinimide (NHS, Sigma-Aldrich), and 0.9 μg ml⁻¹ pAHSA. The solution was then kept at 28° C. with ultra-sonication for 2 h for the cross-linking of pAHSA on CNTs to become CNT-label. Third, the CNT-label was extracted from centrifugation at 9000 rpm for 5 min. The supernatant including excess reagents was then disposed of and the precipitate was re-dispersed in buffer solution. These wash steps were repeated for three times, and the final CNT-label solution was kept in buffer solution with ultra-sonication about 30 min before used.

### HSA Detection

The biosensor fabricated in this work was used to detect the sensing-target HSA with the concentration ranging from 2×10⁻⁷ to 2×10⁻¹ mg ml⁻¹, including one control sample without HSA. First, the sensing-substrate was immersed in HSA solution and shaken at 35° C. for 1 h, followed by the wash in PBS for several times for the specific bonding of HSA on the sensing-substrate. Second, the HSA-bonded sensing-substrate was immersed in a pAHSA modified CNT-label solution under ultra-sonication at 28° C. for 1 h for the bonding of CNT-label onto the detected HSA on the sensing-substrate (CNT-labeled sensing-substrate). The CNT-labeled sensing-substrate was then by rinsed with PBS to remove unbound CNT-label for several times and dried with N₂ flow, followed by the optical transmission measurement by using UV-Vis (Cary 60).

### Result

The biosensor is composed of two components, a sensing substrate and a CNT-label, respectively. The sensing-substrate is made of a piece of glass with the surface modified with bovine serum albumin (BSA, sigma, B2518)/monoclonal anti-human serum albumin (mAHSA, abcam, ab18083)/3-aminopropyltriethoxysilane (APTES, Alfa Aesar, A10668), to provide specific binding to human serum albumin (HSA, abcam, ab67670).

The CNT-label is composed of CNTs which were immobilized with polyclonal AHSA (pAHSA, abcam, ab24207) to label the detected HSA on the sensing-substrate.

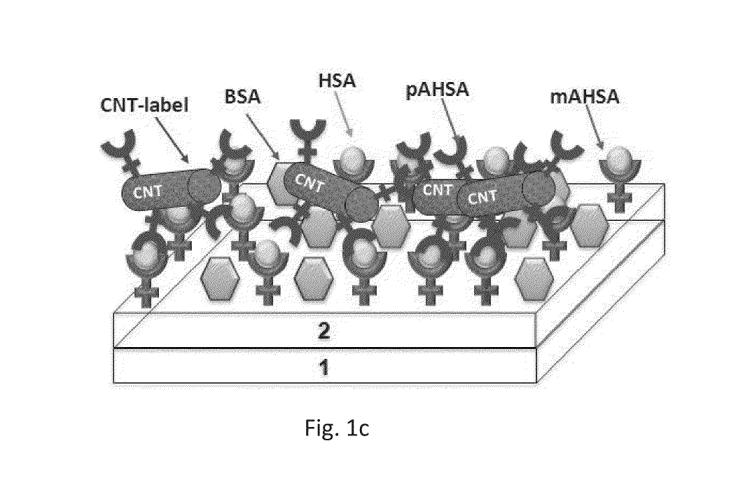

The detection process is briefly described as follows. HSA was immobilized on the sensing-substrate and then bonded with the CNT-label from the final structure, CNT labeled sensing-substrate, whose optical transmission was measured by UV-Vis (Cary 60). To ensure the specific HSA-detection of the biosensor, the sensing-substrate was functionalized with mAHSA and BSA. Therefore, the immobilization of the mAHSA on the APTES modified glass, and the blocking of BSA on untreated and non-specific bonding sites before HSA sensing should be confirmed, as illustrated in FIGS. 2a-2d.

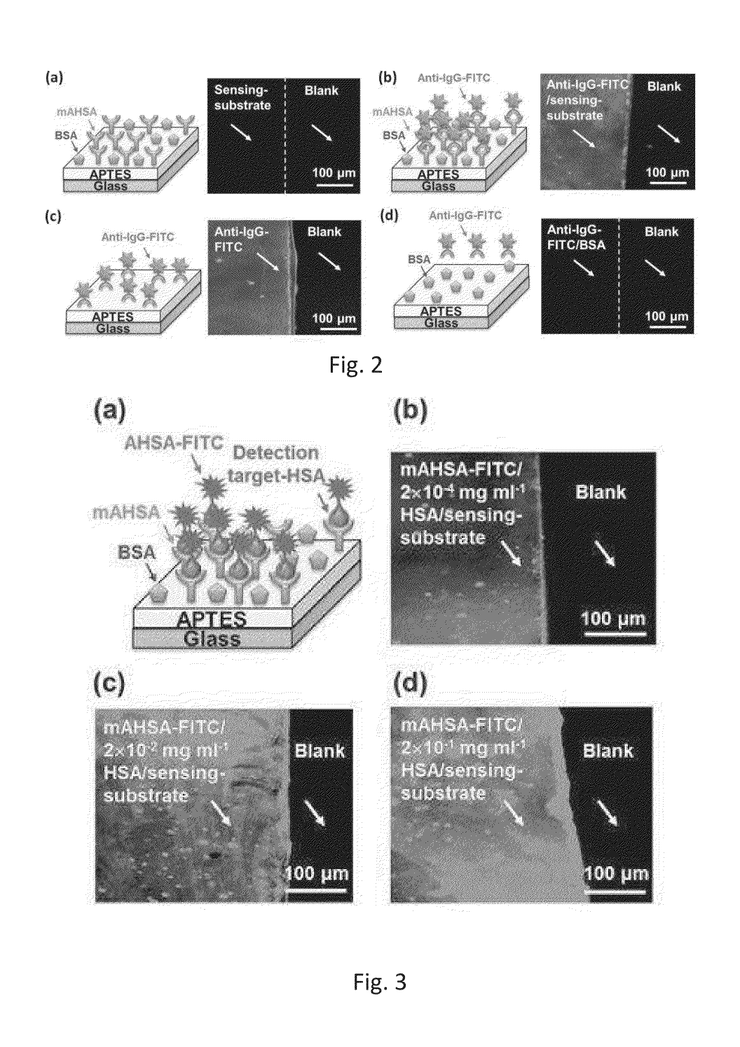

After the various concentrations of HSA were applied on the sensing target, it is important to ensure the success of HSA conjugation with the mAHSA on the sensing-substrate which can be verified by applying rabbit polyclonal to HSA with FITC (AHSA-FITC, abcam, ab34669), and schematics diagram as shown in FIG. 3a. FIGS. 3b-3d show green fluorescence images of the sensing-substrate applied with AHSA-FITC, and after the addition of HSA in concentrations of 2×10⁻⁴, 2×10⁻², and 2×10⁻¹ mg ml⁻¹, respectively. The figures show that the intensity of fluorescence rises with the increase in HSA concentration, indicating that the detection target HSA was conjugated on the sensing-substrate successfully.

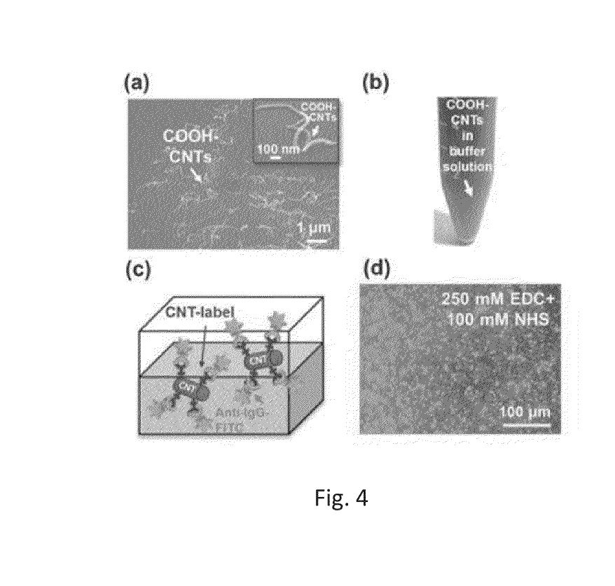

The morphology of COOH-modified CNTs used in this study is shown in the scanning electron microscope (SEM) image in FIG. 4a. The length and diameter of COOH-modified CNTs are 0.5-2 mm and about 20 nm, respectively. In addition, the CNT-label should be stored in buffer solution with pH=7.4 to maintain its activity. To ensure the well-dispersion of COOH-modified CNTs in buffer solution, four common buffer solutions PBS, buffer solution (KH₂PO₄ (0.2 g L⁻¹) and Na₂HPO₄ (1.16 g L⁻¹)), Hank's Balanced Salt Solution (HBSS), and tris(hydroxymethyl)aminomethane hydrochloride (Tris-HCl), were tested in this work. The results indicated that the buffer solution was experimentally verified for better CNT dispersion than others and was used in this work. The COOH-modified CNTs was still dispersed well in buffer solution after being stored statically for 24 h, as shown in FIG. 4b.

As the CNT-label was utilized as a label material for HAS detection, it is important to verify the success of the crosslinking between pAHSA and COOH-CNTs after EDC-NHS reaction. The cross-linking condition can be determined by applying the anti-IgG-FITC onto the CNT-label. The brightest fluorescence image showed that the optimum condition for the crosslinking of pAHSA with COOH-modified CNT was 250 mM EDC and 100 mM NHS, as shown in FIG. 4d.

### Transmission Spectra of the Sensing-Substrate

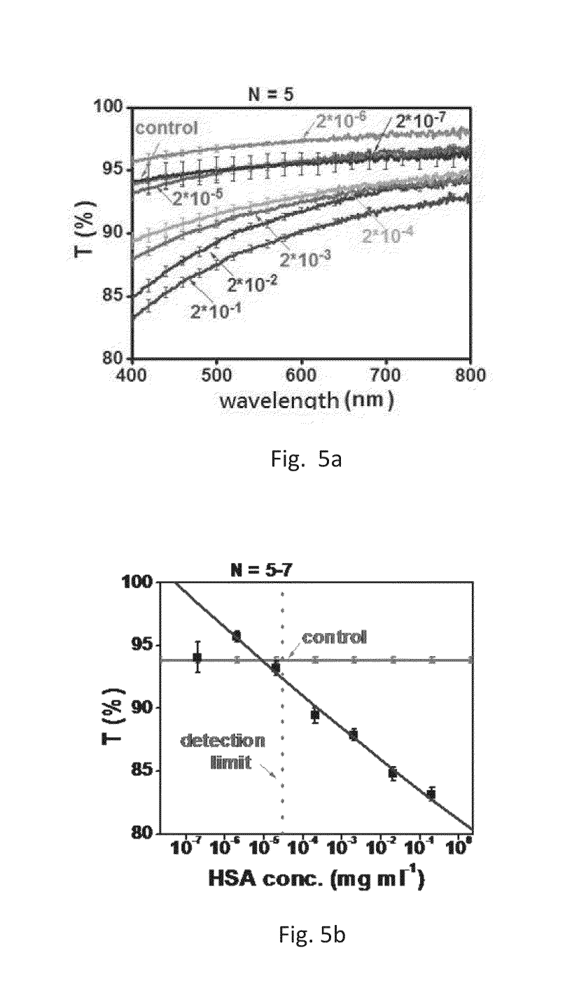

The mean transmission value at 400 nm for the ten specimens was 95.8% and the relative standard deviations (R.S.D.) are less than 0.5%. This indicated that the process of fabrication the sensing-substrate had an excellent reproducibility. Therefore, the transmission value was viewed as a constant within the various sensing-substrate specimens. To characterize the sensing module of the biosensor, the transmission spectra of the biosensor were monitored between process steps, including after APTES modification, after mAHSA and BSA immobilization (sensing-substrate), and HSA conjugation. The spectra showed that the transmission was only significantly reduced after applying the CNT-label on the HSA-bonded sensing-substrate. Accordingly, the single transmission measurement of the biosensor was done at the CNT-labeled sensing-substrate with various HSA concentrations. FIG. 5a showed the transmission spectra of the CNT-labeled sensing-substrate with the HSA concentrations of 0 and 2×10⁻⁷ to 2×10⁻¹ mg ml⁻¹. The CNT-labeled sensing-substrate without HSA (HSA concentration: 0 mg ml⁻¹) served as the control specimen. The measured transmission value of 93.9% was considered as the background level which sets the detection limit of the biosensor demonstrated in this work. It can be observed that there is a consistent reduction of transmission with increasing HSA concentrations. To ensure the reproducibility and consistency of this biosensor, five different samples for each HSA concentration prepared at different time were measured (N=5). The relative standard deviations (R.S.D.) of the transmission spectra measured by UV-Vis shown on FIG. 5c are all less than 2%, which indicates good reproducibility.

Furthermore, to quantify the HSA concentrations, the transmission signals were measured at the wavelength of 400 nm, the most significant changes of the transmission spectra, after CNT-labeled sensing-substrate with the HSA concentrations ranging from 2×10⁻⁷ to 2×10⁻¹ mg ml⁻¹. The reduction of optical transmission is mainly contributed by CNTs bound on the substrates because of their high absorption coefficient and the high scattering ability. As shown in FIG. 5b, the transmission ratio is linear to the HSA concentration, (N=5-7) for each HAS ranging from 2×10⁻⁵ to 2×10⁻¹ mg ml⁻¹ in log-scale, with a corresponding regression equation (log_{y}=1.91-0.01 logₓ, R²=0.988). The horizontal dash line in FIG. 5b shows the average transmission value of the control specimen discussed above. This suggests the detection limit of the biosensor for HSA detection is approximately 3×10⁻⁵ mg ml⁻¹. Compared with other nanostructure-based immunoassay biosensors which also use UV-Vis for detection, this biosensor exhibits higher sensitivity and wider detection range than gold nanostructure biosensors. Besides, this approach provides an innovative mechanism to detect antigen instead of applying SPR properties

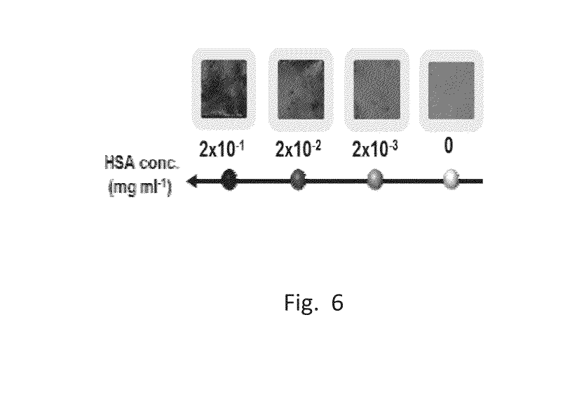

The above results demonstrate the feasibility of using nanostructure material with high absorption and high scattering ability as a label material for biosensor that can quantify antigen concentration, achieve high sensitivity and wide detection range for biosensor application. Moreover, the various transmission values with different HSA concentrations observed in the UV-Vis suggested the feasibility of directly capturing these colorimetric changes by the naked eye for detection. After stacking three same specimens, the color difference at higher concentration of HSA could be observed visually as shown in FIG. 6. This visual result demonstrated that this sensor bares the potential for daily life health check-ups through direct observation by the user, hopefully, as convenient as today's off-the-counter pregnancy test kits.

To sum up, the present invention develops a novel biosensor for HSA detection by utilizing a label material with high absorption coefficient and high light scattering ability. The biosensor using CNT as a label for HSA detection was demonstrated successfully. The calibration results show good linearity between HSA concentration and reduction in optical transmission. The biosensor shows the following advantages. First, the high sensitivity for HSA detection with a detection limit of 3×10⁻⁵ mg ml⁻¹ and wide detection range of 2×10⁻⁵ to 2×10⁻¹ mg ml⁻¹. Second, the cost of the biosensor is reduced by using CNTs as a label material rather than AuNPs. Third, the biosensor could be operated in broad light wavelength range without the limitation of specific wavelength and shows the same performance for detection. Fourth, the colorimetric biosensor could be detected by direct observation, because the color changed with different concentration of HSA.

While the invention can be subject to various modifications and alternative forms, a specific example thereof has been shown in the drawings and is herein described in detail. It should be understood, however, that the invention is not to be limited to the particular form disclosed, but on the contrary, the invention is to cover all modifications, equivalents, and alternatives falling within the spirit and scope of the appended claims.

What is claimed is:

1. An enzyme-free colorimetric immunoassay used for detecting an antigen, comprising:
providing a colorimetric antibody, wherein the colorimetric antibody is coupled with a nanomaterial and not connected with an enzyme, and the nanomaterial is black; and
measuring colorimetric effect of the colorimetric antibody so as to determine the presence or concentration of the antigen.

2. The enzyme-free colorimetric immunoassay as claimed in claim 1, being operated on a solid supporting.

3. The enzyme-free colorimetric immunoassay as claimed in claim 1, being operated on a transparent substrate.

4. The enzyme-free colorimetric immunoassay as claimed in claim 3, wherein the transparent substrate is a glass substrate.

5. The enzyme-free colorimetric immunoassay as claimed in claim 3, wherein the transparent substrate is a polystyrene substrate.

6. The enzyme-free colorimetric immunoassay as claimed in claim 3, wherein the measuring the colorimetric effect of the colorimetric antibody is achieved by measuring the transmittance of the transparent substrate.

7. The enzyme-free colorimetric immunoassay as claimed in claim 3, wherein the measuring the colorimetric effect of the colorimetric antibody is achieved by measuring the absorbance of the transparent substrate at wavelength ranging from 400 nm to 800 nm.

8. The enzyme-free colorimetric immunoassay as claimed in claim 1, wherein the measuring the colorimetric effect of the colorimetric antibody is achieved by naked eye.

9. The enzyme-free colorimetric immunoassay as claimed in claim 1, wherein the measuring the colorimetric effect of the colorimetric antibody is achieved by UV/VIS.

10. The enzyme-free colorimetric immunoassay as claimed in claim 1, wherein the nanomaterial is CNT.

11. The enzyme-free colorimetric immunoassay as claimed in claim 1, wherein the nanomaterial comprises SWCNT or MWCNT.

12. The enzyme-free colorimetric immunoassay as claimed in claim 1, wherein the nanomaterial comprises graphene, Co₃O₄ or WS₂.

13. The enzyme-free colorimetric immunoassay as claimed in claim 1, including competitive method, indirect method or sandwich method.

14. The enzyme-free colorimetric immunoassay as claimed in claim 1, wherein the colorimetric antibody is conjugated to the antigen.

15. The enzyme-free colorimetric immunoassay as claimed in claim 1, further comprising:
providing a primary antibody, wherein the primary antibody is conjugated to the antigen and the colorimetric antibody is conjugated to the primary antibody.
